(19) United States
(12) Patent Application Publication
(10) Pub. No.: US 2024/0415567 A1
(43) Pub. Date: Dec. 19, 2024

(54) RESECTOSCOPE

(71) Applicant: Hangzhou HAWK Optical Electronic Instruments Co., Ltd., Zhejiang (CN)

(72) Inventors: GUOHUA BAO, Zhejiang (CN); LEI WANG, Zhejiang (CN); HUIYI CHEN, Zhejiang (CN); HAI LIN, Zhejiang (CN); YINGCHUN SHAN, Zhejiang (CN); XIAOJING KONG, Zhejiang (CN)

(21) Appl. No.: 18/609,174

(22) Filed: Mar. 19, 2024

(30) Foreign Application Priority Data
Jun. 13, 2023 (CN) 2023106929400

(51) Int. Cl. A61B 18/14

(52) U.S. Cl. CPC A61B 18/149; A61B 18/1485; A61B 2018/1407

(57) ABSTRACT

A resectoscope includes an endoscope assembly, a working element, a sheath assembly, and a resection loop assembly. The working element includes a connecting tube, a connector portion, and a sliding block part including a sliding block body and in sliding connection with the connecting tube. The resection loop assembly includes a connecting rod to connect the sliding block body. An accommodation cavity, a clamping mechanism, and a perforation are provided on the sliding block body, one end of the clamping mechanism disposed in the accommodation cavity, the accommodation cavity and the clamping mechanism hermetically connected, and the perforation in communication with the accommodation cavity; and a first sealing ring is provided on an inner wall of the perforation, a spacing formed between the first sealing ring and the accommodation cavity, one end of the connecting rod, after passing through the first sealing ring, being snap fastened with the clamping mechanism.

### CROSS-REFERENCE TO RELATED APPLICATION AND CLAIM OF PRIORITY

This application claims the benefit under 35 USC § 119 of Chinese Patent Application No. 2023106929400, filed on Jun. 13, 2023, in the China Intellectual Property Office, the entire disclosure of which is incorporated herein by reference for all purposes.

### BACKGROUND

### 1. Technical Field

The disclosure relates to the technical field of medical instruments, and more particularly relates to a resectoscope.

### 2. Background

A resectoscope is a common electrosurgical instrument widely used in combination with electrodes to perform electroresection, a minimally invasive surgical therapy, for treatment of urological diseases such as prostatic hyperplasia, bladder neoplasms, uterine fibroid and endometrial polyps, etc. An existing resectoscope generally includes an endoscopic assembly, a working element, a sheath assembly, and a resection loop assembly, among which the working element is configured to connect the endoscope, the resection loop assembly, and the sheath assembly. When performing a surgery with the existing resectoscope, a surgeon would manipulate the working element to drive the resection loop assembly to move relative to the sheath assembly, with one end of the resection loop assembly extending out of the sheath assembly, whereby a body tissue is resected by the resection loop extending out of the sheath assembly. Since the resection loop assembly is required to move relative to the working element and the sheath assembly, a certain gap needs to be arranged between the resection loop assembly and the working element or sheath assembly to enable sliding of the resection loop assembly.

In addition, the surgical procedure requires injecting normal saline into the sheath assembly, which is highly demanding on sealing performance of the sheath assembly; if the sealing structure does not provide good sealing, the normal saline inside the sheath assembly would exude through the gap to contaminate the exterior structure of the resectoscope, increasing the difficulty of cleaning the resectoscope after the surgery; moreover, the exuded normal saline would also access other positions to cause contamination, increasing infection risk during the surgical procedure; and the exuded normal saline would also easily access an accommodation cavity in a sliding block assembly where the resection loop assembly is connected, causing corrosion to the electrical connection structure in the accommodation cavity.

### SUMMARY

To overcome a drawback of corrosion to an electrical connection structure in an existing resectoscope due to easy exuding of normal saline in a sheath assembly into a sliding block assembly, a first objective of the disclosure is to provide a resectoscope which can prevent external water from accessing the inside of the sliding block body via a resection loop assembly and thus prevent corrosion to the electrical connection structure.

To overcome a drawback of contamination caused by an existing resectoscope due to easy exuding of the normal saline in a sheath assembly and dripping to other positions, a second objective of the disclosure is to provide a resectoscope which can seal an interstice between a resection loop assembly and a working element and thus prevent exuding of the normal saline via the interstice.

To realize the first objective, the disclosure adopts a technical solution set forth infra.

A resectoscope, including an endoscope assembly, a working element, a sheath assembly, and a resection loop assembly, the working element including a connecting tube, a connector portion fixed at one end of the connecting tube, and a sliding block part in sliding connection with the connecting tube, the sliding block part including a sliding block body, the resection loop assembly including a connecting rod configured to connect the sliding block body, wherein an accommodation cavity, a clamping mechanism for securing the connecting rod, and a perforation configured for the connecting rod to pass through are provided on the sliding block body, one end of the clamping mechanism being disposed in the accommodation cavity, the clamping mechanism and the accommodation cavity being hermetically connected, the perforation being in communication with the accommodation cavity; a first sealing ring is provided on an inner wall of the perforation, a spacing being formed between the first sealing ring and the accommodation cavity, one end of the connecting rod, after passing through the first sealing ring, being snap fastened with the clamping mechanism, so that the accommodation cavity is sealed from the outside of the sliding block body, causing shrinkage of volume and increase of gas pressure inside the accommodation cavity.

In this technical solution, the first sealing ring may prevent external water from accessing the sliding block body via the gap between the connecting rod and the perforation, thereby preventing corrosion to the electrical connection structure. During mounting of the resection loop assembly, after one end of the connecting rod is inserted in the perforation and extends through the first sealing ring, since the first sealing ring maintains hermetical connection to the connecting rod, a portion of the connecting rod further inserted in the accommodation cavity shrinks the volume of the accommodation cavity, whereby a pressurized space is formed inside the accommodation cavity, the gas pressure in the accommodation cavity being higher than the atmospheric pressure outside the accommodation cavity, so that even if the sealing performance is currently degraded, the pressurized gas in the accommodation cavity would be released slowly to outside, allowing no external water to access the accommodation cavity, further enhancing the sealing effect. The volume of the above-mentioned accommodation cavity includes a space within the accommodation cavity and a space of the perforation on the inner side of the first sealing ring. In this technical solution, the accommodation cavity communicates with the outside of the sliding block body only via the perforation, and after one end of the resection loop assembly passes through the first sealing ring, the accommodation cavity is completely isolated from the outside of the sliding block body, so that air cannot escape out of the accommodation cavity.

Preferably, one end of the connecting rod is provided with a snap-on connection tip; the clamping mechanism includes a stationary base, a pressing rod, and a movable block, the stationary base and the sliding block body being hermetically secured, the movable block being slidingly sealed with the stationary base and fixed to the pressing rod, the pressing rod being disposed in the accommodation cavity, the perforation being in communication with the accommodation cavity; a snap-fastening element adapted to the snap-on connection tip is securely provided on the pressing rod; a first elastic element is disposed between the stationary base and the movable block so that when the snap-on connection tip is inserted in the clamping mechanism, the snap-fastening element clamps the snap-on connection tip.

This technical solution may realize quick snap-on connection between the resection loop assembly and the sliding block part. In need of connecting the resection loop assembly, the movable block is depressed or pushed to drive the pressing rod to move, and then the snap-on connection tip is inserted in the perforation; after the snap-on connection tip is inserted in place, the movable block is released, and under the action of the first elastic element, the snap-fastening element approaches to clamp the snap-on connection tip, whereby the connecting rod and the sliding block part are fixed. In need of removing the resection loop assembly, the movable block is depressed or pushed to drive the pressing rod to move, and the snap-fastening element moves away from the snap-on connection tip, resulting in disappearance of the snap effect; and now, the connecting rod may be pulled out. In this technical solution, since the stationary base and the sliding block body are hermetically secured and the movable block and the stationary base are slidingly sealed, the accommodation cavity is isolated from the external space, resulting in a good sealing performance.

Preferably, the first elastic element drives the movable block to move towards a side proximal to the accommodation cavity, so that the snap-fastening element presses tightly against the snap-on connection tip, whereby volume in the accommodation cavity is shrunk, which increases gas pressure in the accommodation cavity when the snap-on connection tip is in a state of being inserted in the accommodation cavity.

In this technical solution, during mounting of the resection loop assembly, firstly the movable block is pulled to move towards a side distant from the accommodation cavity; now, the volume inside the accommodation cavity increases, and the gas enters the accommodation cavity via the perforation; after the connecting rod is inserted in the perforation, the accommodation cavity is isolated from the outside; after the snap-on connection tip is inserted in place, the movable block is released, and under the action of the first elastic element, the movable block is reset to move towards a side proximal to the accommodation cavity, so that the snap-fastening element presses tightly against the snap-on connection tip, causing shrinkage of the volume in the accommodation cavity; and now, the gas in the accommodation cavity is further compressed, further enhancing the sealing effect.

Preferably, the pressing rod is electrically connected to a receptacle on the sliding block part via an electrically conductive telescopic spring. This solution may maintain electrical connection of the pressing rod, while moving, to the receptacle in the sliding block part.

Preferably, an end of the snap-on connection tip is provided with a conical guiding surface, so that when the snap-on connection tip is inserted in the clamping mechanism, the conical guiding surface presses tightly against the snap-fastening element, driving the snap-fastening element to slide towards a direction away from the snap-on connection tip.

In this technical solution, when the snap-on connection tip is inserted in the perforation, the conical guiding surface engages the snap-fastening element to press against the snap-fastening element, driving the pressing rod to move; and after the snap-on connection tip is inserted in place, under the action of the first elastic element, the snap-fastening element is automatically reset to clamp the snap-on connection tip.

Preferably, a connecting hole is provided on the pressing rod, the snap-fastening element being disposed on an inner wall of a side of the connecting hole distant from the movable block; when the snap-on connection tip is inserted in the connecting hole, the first elastic element drives the movable block to move towards a side away from the accommodation cavity, so that the snap-fastening element presses tightly against the snap-on connection tip. This solution may realize snap-on connection between the pressing rod and the connecting rod.

Preferably, a mounting hole is provided at one end of the connector portion, an opposite end of the connecting rod passing through the mounting hole and being inserted in the connector portion; a second sealing ring and an urging element are provided on the mounting hole, the second sealing ring being sleeved outside the connecting rod; the urging element is adjustably disposed on the mounting hole to press tightly against the second sealing ring, so that the second sealing ring is deformed to press tightly and hermetically against an underside of the mounting hole and an outer sidewall of the connecting rod.

This technical solution may realize the second objective of the disclosure. The urging element may press tightly against the second sealing ring, causing the second sealing ring to be deformed and press tightly and hermetically against the underside of the mounting hole and the outer sidewall of the connecting rod, thereby sealing an interstice between the connecting rod and the mounting hole, preventing the normal saline from exuding via the interstice; and in addition, the urging element is adjustably disposed on the mounting hole, so that even if the second sealing ring is worn or the sealing performance is degraded, the urging force of the urging element against the second sealing ring can still be enhanced by adjusting the position of the urging element, which increases the deformation amount of the second sealing ring as well as the pressing force against the underside of the mounting hole and the outer sidewall of the connecting rod, thereby enhancing the sealing effect.

Preferably, the urging element and the mounting hole are thread-fitted, a through-hole facilitating the connecting rod to pass through is formed on the urging element, and one end of the urging element distant from the second sealing ring is provided with a recessed groove facilitating a screw driver to turn the urging element; or, the urging element and the mounting hole are detachably connected and fixed via interference-fitting.

In the first parallel technical solution noted supra, the recessed groove facilitates turning of the urging element by a screw driver, and the position of the urging element in the mounting hole may be adjusted by turning the urging element, whereby the pressing force of the urging element against the second sealing ring is adjusted. The second parallel technical solution noted supra may realize position adjustment of the urging element in the mounting hole, thereby adjusting the pressing force of the urging element against the second sealing ring.

Preferably, a regulating element and a delivering element are further provided, the urging element being slidably disposed in the mounting hole, the regulating element being disposed outside the mounting hole and thread-fitted with the connector portion, and the regulating element pressing tightly against the delivering element so that the delivering element presses tightly against the urging element.

In this technical solution, the urging element and the regulating element are arranged in stagger; and the connecting rod is provided at the position of the urging element, which is inconvenient for directly adjusting the position of the urging element. By changing the position of the regulating element, the position of the urging element may be indirectly adjusted, whereby the pressing force of the urging element against the second sealing ring is regulated. In addition, the connecting rod does not obstruct the regulation trajectory, thereby facilitating the regulation.

Preferably, one end of the delivering element is fixed to the urging element.

### The Disclosure Offers the Following Benefits:

(1) the disclosure may prevent external water from accessing the inside of the sliding block body via an interstice between the connecting rod and the perforation, thereby preventing corrosion to the electrical connection structure;
(2) during mounting of the resection loop assembly, after one end of the connecting rod is inserted in the perforation and extends through the first sealing ring, since the first sealing ring maintains hermetical connection to the connecting rod, a portion of the connecting rod further inserted in the accommodation cavity shrinks the volume of the accommodation cavity, whereby a pressurized space is formed inside the accommodation cavity, the gas pressure in the accommodation cavity being higher than the atmospheric pressure outside the accommodation cavity, so that even if the sealing performance is currently degraded, the pressurized gas in the accommodation cavity would be released slowly to the outside, allowing no external water to access the accommodation cavity, further enhancing the sealing effect;
(3) during mounting of the resection loop assembly, firstly the movable block is pulled to move towards a side distant from the accommodation cavity; now, the volume inside the accommodation cavity increases, and the gas enters the accommodation cavity via the perforation; after the connecting rod is inserted in the perforation, the accommodation cavity is isolated from the outside; after the snap-on connection tip is inserted in place, the movable block is released, and under the action of the first elastic element, the movable block is reset to move towards a side proximal to the accommodation cavity, so that the snap-fastening element presses tightly against the snap-on connection tip, causing shrinkage of the volume in the accommodation cavity; and now, the gas in the accommodation cavity is further compressed, further enhancing the sealing effect; and
(4) the urging element may press tightly against the second sealing ring, causing the second sealing ring to be deformed and press tightly and hermetically against the underside of the mounting hole and the outer sidewall of the connecting rod, thereby sealing an interstice between the connecting rod and the mounting hole, preventing the normal saline from exuding via the interstice; and in addition, the urging element is adjustably disposed on the mounting hole, so that even if the second sealing ring is worn or the sealing performance is degraded, the urging force of the urging element against the second sealing ring can still be enhanced by adjusting the position of the urging element, which increases the deformation amount of the second sealing ring as well as the pressing force against the underside of the mounting hole and the outer sidewall of the connecting rod, thereby enhancing the sealing effect.

### BRIEF DESCRIPTION OF THE DRAWINGS

FIG. 1 is a sectional view of a resectoscope according to the disclosure;

FIG. 2 is a local enlarged view of part A in FIG. 1;

FIG. 3 is a local enlarged view of part B in FIG. 1;

FIG. 4 is a first local structural schematic diagram of a working element in the disclosure;

FIG. 5 is a second local structural schematic diagram of the working element in the disclosure;

FIG. 6 is a local structural schematic diagram of embodiment 6; and

FIG. 7 is an axonometric view of the resectoscope according to the disclosure.

### DETAILED DESCRIPTION OF THE EMBODIMENTS

Hereinafter, the disclosure will be described in further detail through specific

embodiments with reference to the accompanying drawings.

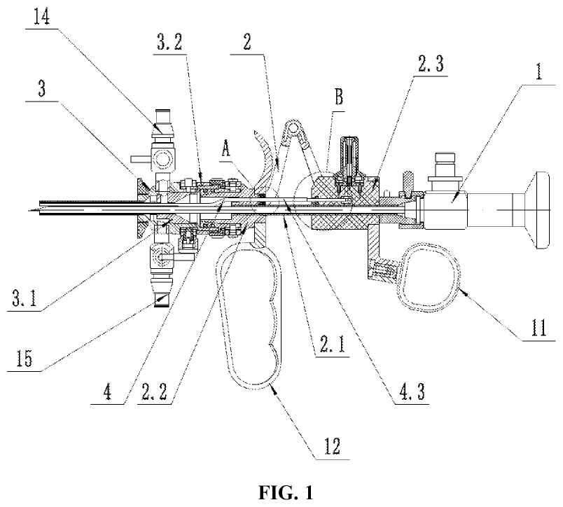

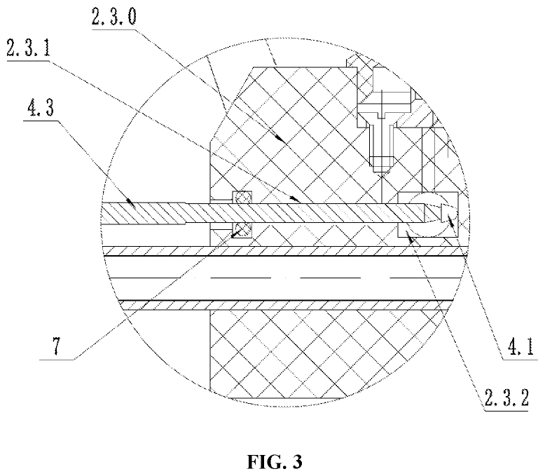

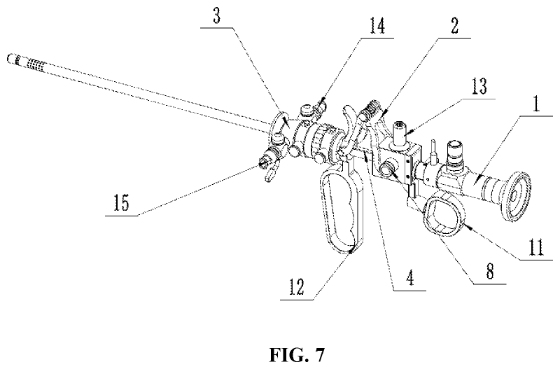

As illustrated in FIGS. 1 to 7, a resectoscope includes an endoscope assembly 1, a working element 2, a sheath assembly 3, and a connecting rod 4.3; the sheath assembly 3 includes an inner sheath 3.1 and an outer sheath 3.2, the outer sheath 3.2 being sleeved outside the inner sheath 3.1, and the outer sheath 3.2 and the inner sheath 3.1 being snap fastened via a snap-fastening structure; the working element 2 includes a connecting tube 2.1, a connector portion 2.2 fixed at one end of the connecting tube 2.1, and a sliding block part 2.3 in sliding connection with the connecting tube 2.1, the endoscope assembly 1 being securely snap fastened to the opposite end of the connecting tube 2.1; one end of the endoscope assembly 1 passes through the connecting tube 2.1 and extends into the inner sheath 3.1 to observe a to-be-resected site; an inflow valve 14 and an outflow valve 15 are arranged on the outer sheath 3.2, an inner space of the inflow valve 14 being in communication with an inner space of the inner sheath 3.1 to enable injection of normal saline, a gap being formed between the inner sheath 3.1 and the outer sheath 3.2, via which gap the normal saline is drained out from the outflow valve; the sheath assembly 3 is securely snap fastened to the connector portion 2.2, an inner space of the sheath assembly 3 being in communication with an inner space of the connector portion 2.2; the sliding block part 2.3 includes a sliding block body 2.3.0; the resection loop assembly 4 includes a connecting rod 4.3 connected to the sliding block body 2.3.0, one end of the connecting rod 4.3 being inserted in the sliding block body 2.3.0 and securely snap fastened to the sliding block body 2.3.0; a receptacle 13 is provided in the sliding block part 2.3, the receptable 13 being configured to electrically connect an external power source, one end of the connecting rod 4.3 being electrically connected to the receptacle 13, and one end of the connector portion 2.2 being provided with a mounting hole 2.2.1; the opposite end of the connecting rod 4.3 passes through the mounting hole 2.2.1, is inserted in the connector portion 2.2, and then passes through the sheath assembly 3 to extend out from the opposite end of the sheath assembly 3; and a finger hook 11 is fixedly attached to the sliding block body 2.3.0, and a finger ring 12 is fixedly attached to the connector portion 2.2.

### Embodiment 1

As illustrated in FIGS. 1 to 4, a resectoscope includes an endoscope assembly 1, a working element 2, a sheath assembly 3, and a resection loop assembly 4; the working element 2 includes a connecting tube 2.1, a connector portion 2.2 fixed at one end of the connecting tube 2.1, and a sliding block part 2.3 in sliding connection with the connecting tube 2.1; one end of the resection loop assembly 4 is inserted into the sliding block body 2.3.0 and fixed to the sliding block body 2.3.0; the sliding block part 2.3 includes a sliding block body 2.3.0; the resection loop assembly 4 includes a connecting rod 4.3 configured to connect the sliding block body 2.3.0; an accommodation cavity 2.3.2, a clamping mechanism 8 for fixing the connecting rod 4.3, and a perforation 2.3.1 configured for the connecting rod 4.3 to pass through are provided on the sliding block body 2.3.0, one end of the clamping mechanism 8 being disposed in the accommodation cavity 2.3.2, the clamping mechanism 8 and the accommodation cavity 2.3.2 being hermetically connected, the perforation 2.3.1 being in communication with the accommodation cavity 2.3.2, the accommodation cavity 2.3.2 being sealed from the outside of the sliding block body 2.3.0, a first sealing ring 7 being provided on an inner wall of the perforation 2.3.1, so that when the connecting rod 4.3 passes through the first sealing ring 7, they are hermetically connected; a spacing is provided between the first sealing ring 7 and the accommodation cavity 2.3.2; one end of the connecting rod 4.3, after passing through the first sealing ring 7, is snap fastened with the clamping mechanism 8, causing reduction of the volume and increase of the gas pressure in the accommodation cavity 2.3.2.

In the technical solution noted supra, the first sealing ring 7 may prevent external water from accessing the sliding block body 2.3.0 via the gap between the connecting rod 4.3 and the perforation 2.3.1, thereby preventing corrosion to the electrical connection structure. During mounting of the resection loop assembly 4, after one end of the connecting rod 4.3 is inserted in the perforation 2.3.1 and extends through the first sealing ring 7, since the first sealing ring 7 maintains hermetical connection to the connecting rod 4.3, a portion of the connecting rod 4.3 further inserted in the accommodation cavity 2.3.2 shrinks the volume of the accommodation cavity 2.3.2, whereby a pressurized space is formed inside the accommodation cavity 2.3.2, the gas pressure in the accommodation cavity 2.3.2 being higher than the atmospheric pressure outside the accommodation cavity 2.3.2, so that even if the sealing performance is currently degraded, the pressurized gas in the accommodation cavity 2.3.2 would be released slowly to the outside, allowing no external water to access the accommodation cavity 2.3.2, further enhancing the sealing effect. The volume of the above-mentioned accommodation cavity 2.3.2 includes a space within the accommodation cavity 2.3.2 and a space of the perforation 2.3.1 on the inner side of the first sealing ring 7. In this technical solution, the accommodation cavity communicates with the outside of the sliding block body only via the perforation, and after one end of the resection loop assembly passes through the first sealing ring, the accommodation cavity is completely isolated from the outside of the sliding block body, so that air cannot escape out of the accommodation cavity.

It is understood that, in one embodiment, an annular-shaped accommodation groove is formed on an inner wall of the perforation 2.3.1, the first sealing ring 7 being mounted in the annular-shaped accommodation groove, preventing the first sealing ring 7 from moving along an axial direction of the perforation 2.3.1.

It is understood that, in another embodiment, a stepped hole is provided at an outer end of the perforation, a circlip is snap fastened on a sidewall of the stepped hole, the first sealing ring 7 being disposed between the circlip and the underside of the stepped hole, whereby the first sealing ring 7 is fixed in the perforation via the circlip and the underside of the stepped hole, preventing the first sealing ring 7 from moving along the axial direction of the perforation 2.3.1.

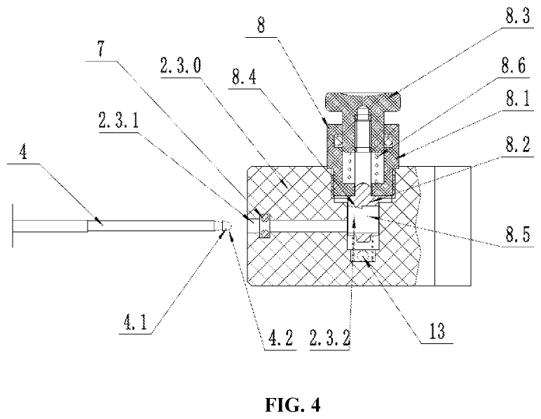

Preferably, as illustrated in FIG. 4, one end of the connecting rod 4.3 is provided with a snap-on connection tip 4.1; the clamping mechanism 8 includes a stationary base 8.1, a pressing rod 8.2, and a movable block 8.3; the stationary seat 8.1 and the sliding block body 2.3.0 are hermetically fixed, the movable block 8.3 being slidingly sealed with the stationary base 8.1 and secured to an outer end of the pressing rod 8.2; the pressing rod 8.2 is inserted in the accommodation cavity 2.3.2, the perforation 2.3.1 communicating with the accommodation cavity 2.3.2; a snap-fastening element 8.4 adapted to the snap-on connection tip 4.1 is provided on the pressing rod 8.2; and a first elastic element 8.6 is disposed between the stationary base 8.1 and the movable block 8.3 so that when the snap-on connection tip 4.1 is inserted in the clamping mechanism 8, under the elastic force of the first elastic element, the snap-fastening element 8.4 maintains a state of tightly clamping the snap-on connection tip 4.1.

This technical solution may realize quick snap-on connection between the connecting rod 4.3 and the sliding block part 2.3. In need of connecting the resection loop assembly 4, the movable block 8.3 is depressed or pushed to drive the pressing rod 8.2 to move, and then the snap-on connection tip 4.1 is inserted in the perforation 2.3.1; after the snap-on connection tip 4.1 is inserted in place, the movable block 8.3 is released, and under the action of the first elastic element 8.6, the snap-fastening element 8.4 approaches to clamp the snap-on connection tip 4.1, whereby the connecting rod 4.3 and the sliding block part 2.3 are fixed. In need of removing the resection loop assembly 4, the movable block 8.3 is depressed or pushed to drive the pressing rod 8.2 to move, and the snap-fastening element 8.4 moves away from the snap-on connection tip 4.1, resulting in disappearance of the snap effect; and now, the connecting rod 4.3 may be pulled out. In this technical solution, since the stationary base 8.1 and the sliding block body 2.3.0 are hermetically secured and the movable block 8.3 and the stationary base 8.1 are slidingly sealed, the accommodation cavity 2.3.2 is isolated from the external space, resulting in a good sealing performance. When the snap-on connection tip 4.1 is inserted in the clamping mechanism 8, the snap-fastening element 8.4 clamps the snap-on connection tip 4.1 tightly. Since the stationary base 8.1 and the clamping mechanism 8 are hermetically secured and the movable block 8.3 and the stationary base 8.1 are slidingly sealed, the accommodation cavity 2.3.2 is isolated from the external space, which may prevent external water from accessing the accommodation cavity 2.3.2.

It is understood that, in another embodiment, a connecting hole 8.5 is formed on the pressing rod 8.2, and the snap-fastening element 8.4 is disposed on an inner wall of the side of the connecting hole 8.5 distant from the movable block 8.3; when the snap-on connection tip 4.1 is inserted in the connecting hole 8.5, the first elastic element 8.6 drives the movable block 8.3 to move towards the side distant from the accommodation cavity 2.3.2, causing the snap-fastening element 8.4 to press tightly against the snap-on connection tip 4.1. This solution can realize snap-on connection between the pressing rod 8.2 and the connecting rod 4.3.

### Embodiment 2

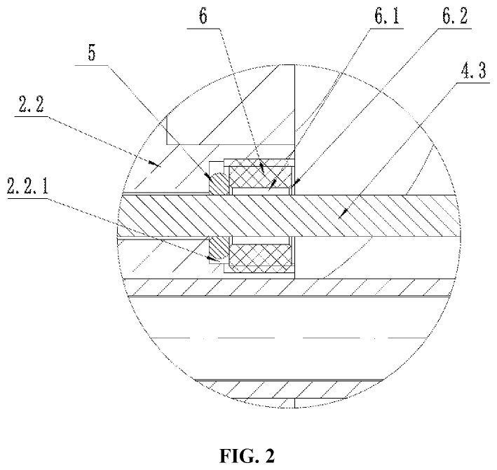

As illustrated in FIG. 1 and FIG. 2, a resectoscope includes an endoscope assembly 1, a working element 2, a sheath assembly 3, and a resection loop assembly 4; the working element 2 includes a connecting tube 2.1, a connector portion 2.2 fixed at one end of the connecting tube 2.1, and a sliding block part 2.3 in sliding connection with the connecting tube 2.1; the sliding block part 2.3 includes a sliding block body 2.3.0; the resection loop assembly 4 includes a connecting rod 4.3 configured to connect the sliding block body 2.3.0, one end of the connecting rod 4.3 being inserted in the sliding block body 2.3.0 and securely snap fastened to the sliding block body 2.3.0, one end of the connector portion 2.2 being formed with a mounting hole 2.2.1, and the opposite end of the connecting rod 4.3 passing through the mounting hole 2.2.1 and extending into the connector portion 2.2; a second sealing ring 5 and an urging element 6 are disposed on the mounting hole 2.2.1, the second sealing ring 5 being sleeved outside the connecting rod 4.3, the urging element 6 being adjustably disposed on the mounting hole 2.2.1 to tightly press against the second sealing ring 5, causing the second sealing ring 5 to be deformed and press tightly and hermetically against the underside of the mounting hole 2.2.1 and an outer sidewall of the connecting rod 4.3.

In this technical solution, one end of the connecting rod 4.3 is securely attached to the sliding block part 2.3, and the opposite end of the connecting rod 4.3 passes through the sheath assembly 3 and extends out from the opposite end of the sheath assembly 3. When performing a surgery with the resectoscope, a surgeon manipulates, via a finger hook 11 and a finger ring 12 on the working element 2, the sliding block part 2.3 to move relative to the connector portion 2.2, so that one end of resection loop assembly 4 extends out of the sheath assembly 3, whereby a body tissue is resected by the resection loop assembly 4 extending out of the sheath assembly 3. During the above-mentioned surgical process, normal saline is injected into the sheath assembly 3; the resection loop assembly 4 moves relative to the connector portion 2.2, i.e., the connecting rod 4.3 slides reciprocally in the mounting hole 2.2.1; and the opposite end of the mounting hole 2.2.1 is in communication with the inner space of the sheath assembly 3. The normal saline in the sheath assembly 3 likely exudes through an interstice between the connecting rod 4.3 and the mounting hole 2.2.1, which would contaminate the outer structure of the resectoscope and cause the resectoscope more difficult to clean after the surgery; and in addition, the normal saline likely drips to other positions, causing contaminations and increasing the infection risk during the surgical process. The urging element 6 may press tightly against the second sealing ring 5, causing the second sealing ring 5 to be deformed and press tightly and hermetically against the underside of the mounting hole 2.2.1 and the outer sidewall of the connecting rod 4.3, thereby sealing an interstice between the connecting rod 4.3 and the mounting hole 2.2.1, preventing the normal saline from exuding via the interstice; in addition, the urging element 6 is adjustably disposed on the mounting hole 2.2.1, so that even if the second sealing ring 5 is worn or the sealing performance is degraded, the urging force of the urging element 6 against the second sealing ring 5 can still be enhanced by adjusting the position of the urging element 6, which increases the deformation amount of the second sealing ring 5 as well as the pressing force against the underside of the mounting hole 2.2.1 and the outer sidewall of the connecting rod 4.3, thereby enhancing the sealing effect.

Preferably, as illustrated in FIGS. 1, 3, and 4, a perforation 2.3.1 configured for the connecting rod 4.3 to pass through is formed on the sliding block body 2.3.0, and a first sealing ring 7 in sealed connection with the connecting rod 4.3 is hermetically attached on an inner wall of the perforation 2.3.1. The first sealing ring 7 may prevent external water from accessing the inside of the sliding block body 2.3.0 via the connecting rod 4.3, thereby preventing corrosion to the electrical connection structure.

Preferably, as illustrated in FIGS. 1, 3, and 4, an accommodation cavity 2.3.2 and a clamping mechanism 8 for securing the connecting rod 4.3 are provided on the sliding block body 2.3.0; one end of the connecting rod 4.3 is provided with a snap-on connection tip 4.1; the clamping mechanism 8 includes a stationary base 8.1, a pressing rod 8.2, and a movable block 8.3, the stationary base 8.1 and the sliding block body 2.3.0 being hermetically secured, and the movable block 8.3 being slidingly sealed with the stationary base 8.1 and secured with the pressing rod 8.2; the pressing rod 8.2 is disposed in the accommodation cavity 2.3.2; the perforation 2.3.1 is in communication with the accommodation cavity 2.3.2; a snap-fastening element 8.4 adapted to the snap-on connection tip 4.1 is securely provided on the pressing rod 8.2; a first elastic element 8.6 is disposed between the stationary base 8.1 and the movable block 8.3 so that when the snap-on connection tip 4.1 is inserted in the clamping mechanism 8, the snap-fastening element 8.4 clamps the snap-on connection tip 4.1.

This technical solution may realize quick snap-on connection between the connecting rod 4.3 and the sliding block part 2.3. In need of connecting the resection loop assembly 4, the movable block 8.3 is depressed or pushed to drive the pressing rod 8.2 to move, and then the snap-on connection tip 4.1 is inserted in the perforation 2.3.1; after snap-on connection tip 4.1 is inserted in place, the movable block 8.3 is released, and under the action of the first elastic element 8.6, the snap-fastening element 8.4 approaches to clamp the snap-on connection tip 4.1, whereby the connecting rod 4.3 is secured to the sliding block part 2.3. In need of removing the resection loop assembly 4, the movable block 8.3 is depressed or pushed to drive the pressing rod 8.2 to move, so that the snap-fastening element 8.4 moves away from the snap-on connection tip 4.1, resulting in disappearance of the snap effect; and now, the connecting rod 4.3 may be pulled out. The first elastic element 8.6 may be a telescopic spring.

Preferably, the pressing rod 8.2 is electrically connected to a receptacle 13 on the sliding block part 2.3 via an electrically conductive telescopic spring made of a metallic material. This solution may ensure that the pressing rod 8.2, while moving, maintains electrical connection to the receptacle 13 on the sliding block part 2.3.

Preferably, after the snap-on connection tip 4.1 is inserted in the perforation 2.3.1 and extends through the first sealing ring 7, the outer sidewall of the connecting rod 4.3 maintains hermetical connection to the first sealing ring 7.

In this technical solution, since the stationary base 8.1 is hermetically secured with the sliding block body 2.3.0 and the movable block 8.3 is slidingly sealed with the stationary base 8.1, the accommodation cavity 2.3.2 is isolated from the external space, which may prevent external water from accessing the accommodation cavity 2.3.2; moreover, during the mounting process of the resection loop assembly 4, since the first sealing ring 7 maintains hermetical connection with the connecting rod 4.3 after the snap-on connection tip 4.1 is inserted in the perforation 2.3.1 and extends through the first sealing ring 7, the portion of the connecting rod 4.3 inserted in the perforation 2.3.1 would shrink the volume of the accommodation cavity 2.3.2, whereby a pressurized space is formed in the accommodation cavity 2.3.2, a gas pressure in the accommodation cavity 2.3.2 being higher than the atmospheric pressure outside the accommodation cavity 2.3.2, so that even if the sealing performance is degraded, the pressurized gas in the accommodation cavity would be released slowly to outside, allowing no external water to access the accommodation cavity 2.3.2, further enhancing the sealing effect. The volume of the above-mentioned accommodation cavity 2.3.2 includes a space inside the accommodation cavity 2.3.2 and a space of the perforation 2.3.1 on the inner side of the first sealing ring 7.

### Embodiment 3

As illustrated in FIG. 4, based on embodiment 1, a conical guiding surface 4.2 is

provided at an end of the snap-on connection tip 4.1; when the snap-on connection tip 4.1 is inserted in the clamping mechanism 8, the conical guiding surface 4.2 presses tightly against the snap-fastening element 8.4, driving the snap-fastening element 8.4 to slide towards a direction away from the snap-on connection tip 4.1.

In this technical solution, when the snap-on connection tip 4.1 is inserted in the perforation 2.3.1, the conical guiding surface 4.2 engages the snap-fastening element 8.4 to press against the snap-fastening element 8.4, driving the pressing rod 8.2 to move; after the snap-on connection tip 4.1 is inserted in place, under the action of the first elastic element 8.6, the snap-fastening element 8.4 is automatically reset to clamp the snap-on connection tip 4.1.

### Embodiment 4

As illustrated in FIG. 4, based on embodiment 1, the first elastic element 8.6 drives the movable block 8.3 to move towards a side proximal to the accommodation cavity 2.3.2, so that the snap-fastening element 8.4 presses tightly against the snap-on connection tip 4.1, causing shrinkage of the volume inside the accommodation cavity 2.3.2.

In this technical solution, during mounting of the resection loop assembly 4, firstly the movable block 8.3 is pulled to move towards a side distant from the accommodation cavity 2.3.2; now, the volume inside the accommodation cavity 2.3.2 increases, and the gas enters the accommodation cavity 2.3.2 via the perforation 2.3.1; after the connecting rod 4.3 is inserted in the perforation 2.3.1, the accommodation cavity 2.3.2 is isolated from the outside; after the snap-on connection tip 4.1 is inserted in place, the movable block 8.3 is released, and under the action of the first elastic element 8.6, the movable block 8.3 is reset to move towards a side proximal to the accommodation cavity 2.3.2, so that the snap-fastening element 8.4 presses tightly against the snap-on connection tip 4.1, causing shrinkage of the volume in the accommodation cavity 2.3.2; now, the gas in the accommodation cavity 2.3.2 is further compressed, further enhancing the sealing effect. The elastic force of the first elastic element 8.6 should be greater than the reactive force generated by the pressurized gas in the accommodation cavity 2.3.2, so as to ensure reliability of the snap fastening structure.

### Embodiment 5

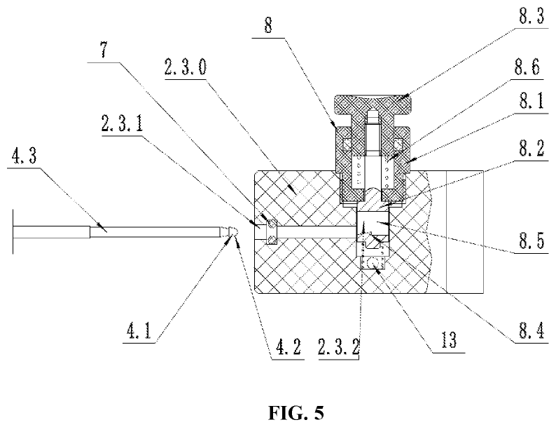

As illustrated in FIG. 5, based on embodiment 1, a connecting hole 8.5 is formed on the pressing rod 8.2, and the snap-fastening element 8.4 is disposed on an inner wall of the side of the connecting hole 8.5 distant from the movable block 8.3; when the snap-on connection tip 4.1 is inserted in the connecting hole 8.5, the first elastic element 8.6 drives the movable block 8.3 to move towards the side distant from the accommodation cavity 2.3.2, so that the snap-fastening element 8.4 presses tightly against the snap-on connection tip 4.1. This solution may realize snap-on connection between the pressing rod 8.2 and the connecting rod 4.3.

### Embodiment 6

As illustrated in FIG. 2, based on embodiment 1, in an embodiment, the urging element 6 and the mounting hole 2.2.1 are thread-fitted, and a through hole 6.1 facilitating the connecting rod 4.3 to pass through is formed on the urging element 6. In this technical solution, a position of the urging element 6 in the mounting hole 2.2.1 may be adjusted by turning the urging element 6, whereby pressing force of the urging element 6 against the second sealing ring 5 is adjusted.

Preferably, a recessed groove 6.2 facilitating a screw driver to turn the urging element 6 is provided at an end of the urging element 6 distant from the second sealing ring 5. The recessed groove 6.2 facilitates the screw driver to turn the urging element 6.

It is understood that, in another embodiment, the urging element and the mounting hole are detachably connected and fixed via interference-fitting This technical solution enables position adjustment of the urging element in the mounting hole, by which the pressing force of the urging element against the second sealing ring is adjusted.

### Embodiment 7

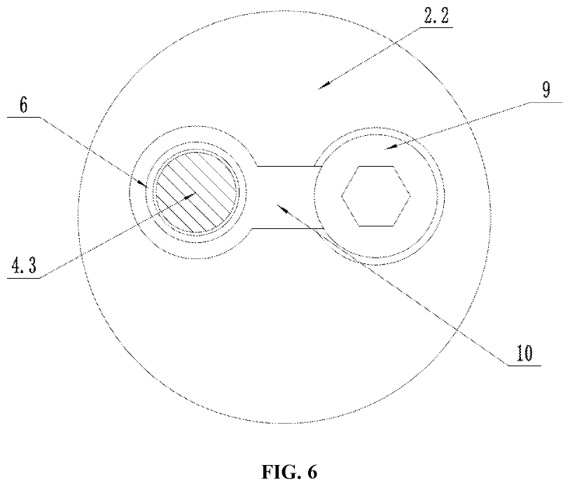

As illustrated in FIG. 6, based on embodiment 1, a regulating element 9 and a delivering element 10 are further provided; the urging element 6 is slidably disposed in the mounting hole 2.2.1; the regulating element 9 is disposed on the outer side of the mounting hole 2.2.1 and in thread-fitting with the connector portion 2.2; and the regulating element 9 presses tightly against the delivering element 10 so that the delivering element 10 presses tightly against the urging element 6.

In this technical solution, the urging element 6 and the regulating element 9 are arranged in stagger. The connecting rod 4.3 is disposed at the position of the urging element 6, which is inconvenient for directly adjusting the position of the urging element 6. By changing the position of the regulating element 9, the position of the urging element 6 may be indirectly adjusted, whereby the pressing force of the urging element 6 against the second sealing ring 5 is regulated. In addition, the connecting rod does not obstruct the regulation trajectory, thereby facilitating the regulation.

Preferably, the urging element 6 is fixed at one end of the delivering element 10.

Finally, it is noted that, the embodiments provided supra are only used to explain the technical solutions of the disclosure, not for limiting them. Although the disclosure has been illustrated in detail with reference to the above embodiments, a person of normal skill in the art should appreciate that, the technical solutions described in the above embodiments may be still modified, or, all or part of the technical features therein may be subjected to equivalent substitution, while such modifications or substitutions would not cause the essence of corresponding technical solutions to depart from the scope of respective technical solutions in the embodiments of the disclosure.

What is claimed is:

1. A resectoscope comprising:
an endoscope assembly;
a working element comprising a connecting tube, a connector portion fixed at one end of the connecting tube, and a sliding block part in sliding connection with the connecting tube, the sliding block part comprising a sliding block body;
a sheath assembly; and
a resection loop assembly comprising a connecting rod configured to connect the sliding block body,
wherein an accommodation cavity, a clamping mechanism for securing the connecting rod, and a perforation configured for the connecting rod to pass through are provided on the sliding block body, one end of the clamping mechanism being disposed in the accommodation cavity, the clamping mechanism and the accommodation cavity being hermetically connected, and the perforation being in communication with the accommodation cavity; a first sealing ring is provided on an inner wall of the perforation, a spacing being formed between the first sealing ring and the accommodation cavity, one end of the connecting rod, after passing through the first sealing ring, being snap fastened with the clamping mechanism, so that the accommodation cavity is sealed from the outside of the sliding block body, causing shrinkage of volume and increase of gas pressure inside the accommodation cavity,
wherein one end of the connecting rod is provided with a snap-on connection tip; the clamping mechanism comprises a stationary base, a pressing rod, and a movable block, the stationary base and the sliding block body being hermetically secured, the movable block being slidingly sealed with the stationary base and fixed to the pressing rod, the pressing rod being disposed in the accommodation cavity, the perforation being in communication with the accommodation cavity; a snap-fastening element adapted to the snap-on connection tip is securely provided on the pressing rod; a first elastic element is disposed between the stationary base and the movable block so that when the snap-on connection tip is inserted in the clamping mechanism, the snap-fastening element clamps the snap-on connection tip; the first elastic element drives the movable block to move towards a side proximal to the accommodation cavity, so that the snap-fastening element presses tightly against the snap-on connection tip, whereby the volume in the accommodation cavity is shrunk, which increases the gas pressure in the accommodation cavity when the snap-on connection tip is in a state of being inserted in the accommodation cavity, and
wherein during mounting the resection loop assembly, firstly the movable block is pulled to move towards a side distant from the accommodation cavity, which increases the volume of the accommodation cavity, allowing a gas to enter the accommodation cavity via the perforation; after the snap-on connection tip is inserted in place, the movable block is released; and under the action of the first elastic element, the movable block is reset to move towards a side proximal to the accommodation cavity, so that the snap-fastening element presses tightly against the snap-on connection tip, causing shrinkage of the volume in the accommodation cavity.

2. The resectoscope according to claim 1, wherein the pressing rod is electrically connected to a receptacle on the sliding block part via an electrically conductive telescopic spring.

3. The resectoscope according to claim 1, wherein an end of the snap-on connection tip is provided with a conical guiding surface, so that when the snap-on connection tip is inserted in the clamping mechanism, the conical guiding surface presses tightly against the snap-fastening element, driving the snap-fastening element to slide towards a direction away from the snap-on connection tip.

4. The resectoscope according to claim 1, wherein a mounting hole is provided at one end of the connector portion, an opposite end of the connecting rod passing through the mounting hole and being inserted in the connector portion; a second sealing ring and an urging element are provided on the mounting hole, the second sealing ring being sleeved outside the connecting rod; the urging element is adjustably disposed on the mounting hole to press tightly against the second sealing ring, so that the second sealing ring is deformed to press tightly and hermetically against an underside of the mounting hole and an outer sidewall of the connecting rod.

5. The resectoscope according to claim 4, wherein the urging element and the mounting hole are thread-fitted, a through-hole facilitating the connecting rod to pass through is formed on the urging element, and an end of the urging element distant from the second sealing ring is provided with a recessed groove facilitating a screw driver to turn the urging element; or, the urging element and the mounting hole are detachably connected and fixed via interference-fitting.

6. The resectoscope according to claim 4, wherein a regulating element and a delivering element are further provided, the urging element being slidably disposed in the mounting hole, the regulating element being disposed outside the mounting hole and thread-fitted with the connector portion, and the regulating element pressing tightly against the delivering element so that the delivering element presses tightly against the urging element.

7. The resectoscope according to claim 6, wherein one end of the delivering element is fixed to the urging element.
